(19) United States
(12) Patent Application Publication
(10) Pub. No.: US 2022/0309388 A1
(43) Pub. Date: Sep. 29, 2022

(54) Systems and Methods for Classification and Time Series Calibration in Identity Health Analysis

(71) Applicant: Allstate Insurance Company, Northbrook, IL (US)

(72) Inventors: Dongmin Liu, Libertyville, IL (US); Giang Phan, Queen Creek, AZ (US); Marcus Waldman, Montclair, NJ (US); Akmal Inoyatov, Phoenix, AZ (US); Lindes Roets, Northbrook, IL (US); Jeremy Miller, Hendersonville, TN (US); Kevin Goulet, Northbrook, IL (US)

(21) Appl. No.: 17/215,558

(22) Filed: Mar. 29, 2021

(51) Int. Cl. G06N 20/00; G06K 9/62

(52) U.S. Cl. CPC G06N 20/00; G06K 9/623; G06K 9/6257; G06K 9/6277; G06K 9/6298

(57) ABSTRACT

Aspects of the disclosure relate to using machine learning methods for identity health scoring. A computing platform may train a machine learning model, using historical event information, by: 1) classifying the historical event information using logical regression, and 2) after classifying the historical event information, performing time series calibration on the classified historical event information, wherein training the machine learning model configures the machine learning model to output identity health information. The computing platform may receive new event information. The computing platform may input the new event information into the machine learning model, which may cause the machine learning model to output the identity health information. The computing platform may send, to a client device, the identity health information and one or more commands directing the client device to display an identity health interface, which may cause the client device to display the identity health interface.

### BACKGROUND

Aspects of the disclosure relate to processing systems. In particular, aspects of the disclosure relate to processing systems that train and apply machine learning models.

In some instances, enterprise organizations may use machine learning models to perform regression in time series events for continuous numerical data (e.g., stock market prices). In may be difficult, however, to apply such techniques to categorical data. Accordingly, enterprise organizations affiliated with such categorical data may be unable to apply machine learning models to perform regression in time series events. This inability may limit or otherwise impact the insights that such enterprise organizations are enabled to produce.

### SUMMARY

Aspects of the disclosure provide effective, efficient, scalable, and convenient technical solutions that address and overcome the technical problems associated with identity health scoring.

In accordance with one or more embodiments, a computing platform comprising at least one processor, a communication interface communicatively coupled to the at least one processor, and memory may train a machine learning model, using historical event information, by: 1) classifying the historical event information using logical regression, and 2) after classifying the historical event information, performing time series calibration on the classified historical event information, which may configure the machine learning model to output identity health information. The computing platform may receive new event information. The computing platform may input the new event information into the machine learning model, which may cause the machine learning model to output the identity health information. The computing platform may send, to a client device, the identity health information and one or more commands directing the client device to display an identity health interface, which may cause the client device to display the identity health interface.

In one or more instances, the computing platform may perform the time series calibration by performing one or more of alert compounding, N-day window alert capture, or tri-label encoding. In one or more instances, the computing platform may perform the alert compounding by considering an alert type a number of times that it appears within a capture window.

In one or more examples, the computing platform may perform the N-day window alert capture by considering historical event information from N-days prior to a current date up to the current date. In one or more instances, the historical event information may include identity threat alerts.

In one or more instances, the computing platform may perform the tri-label encoding by: 1) labeling the historical event information based on user input indicating that an identity threat alert correctly identified a threat or incorrectly identified a threat, or 2) labeling the historical event information to indicate that user input was not received for the corresponding alert. In one or more instances, the computing platform may classify the historical event information by: 1) graphing alert data and fraud event data against time, and 2) deriving, based on the graph, identity health score data.

In one or more arrangements, the computing platform may display the identity health interface by displaying the graph. In one or more instances, the computing platform may display the identity health interface by displaying a color coded scale and a user's position on the color coded scale, where the user's position on the color coded scale indicates an identity health status for the user.

In one or more instances, the machine learning model may be configured to provide real time identity health scoring information. In one or more instances, the computing platform may train, using neural network regression, a predictive identity health scoring model configured to predict identity threat events.

These features, along with many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIGS. 1A and 1B depict an illustrative computing environment for applying machine learning for identity health scoring in accordance with one or more example embodiments;

FIGS. 2A-2C depict an illustrative event sequence for applying machine learning for identity health scoring in accordance with one or more example embodiments;

FIG. 3 depicts an illustrative method for applying machine learning for identity health scoring in accordance with one or more example embodiments; and

FIGS. 4 and 5 depict illustrative graphical user interfaces for applying machine learning for identity health scoring in accordance with one or more example embodiments.

### DETAILED DESCRIPTION

In the following description of various illustrative embodiments, reference is made to the accompanying drawings, which form a part hereof, and in which is shown, by way of illustration, various embodiments in which aspects of the disclosure may be practiced. It is to be understood that other embodiments may be utilized, and structural and functional modifications may be made, without departing from the scope of the present disclosure.

It is noted that various connections between elements are discussed in the following description. It is noted that these connections are general and, unless specified otherwise, may be direct or indirect, wired or wireless, and that the specification is not intended to be limiting in this respect.

As a brief introduction to the concepts described further below, systems and methods for identity health scoring (e.g., providing a metric that indicates a likelihood of identity theft) are described. For example, an identity score may be generated based on risk information from alerts (e.g., fraud alerts) and/or user profiles. The system and method may use machine learning techniques to generate an identity health score. More specifically, the systems and methods described herein may use a time series machine learning classifier in a two-step process. For example, a classification model may be trained, and subsequently inserted back into a time series for a time series calibration session.

Standard machine learning may be broken down into four steps: business understanding, model selection, model training, and model deployment. Time series machine learning algorithms include recurrent neural network (RNN) and long short-term memory (LSTM).

The current state of the art has machine learning models that perform regression in time series events used for stock market prediction. Techniques such as RNN and LSTM create artificial memory by feed-forwarding data to show the importance of prior data in a time series event. Most of these techniques work on numerical data that is continuous (e.g., stock market price). Very few, however, have been developed for categorical data.

Categorical data has been used in classification machine learning. There are many commonly used standard algorithms. One is logistic regression, and it may be used in a classification problem to predict true or false, and a probability between zero and one indicating whether or not the prediction is correct. The threshold for a positive classification is normally set between greater than 0.5 and less than 1.

Accordingly, described herein are arrangements for developing a score that indicates risk of adverse outcomes (e.g., stolen identity theft, dark web sale of credentials, email compromise, social media compromise, or the like) that is based on machine learning. Some example aspects to be considered include: 1) the time series problem requires scoring in real time, 2) it is based on discrete numerous types of alert data from monitoring agencies or partners, and 3) the goal is to provide risk scoring based on the idea of financial or compromise health, machine learning is based on user data which is scalable. In light of these aspects, the disclosure herein aims to address the following technical challenges: 1) there are few time series machine learning algorithms currently in existence that can handle discrete categorical data in real time, and most of these tend to be on the research side, 2) scant amounts of alert data for each user makes machine learning difficult, and 3) developing a scoring risk model based on machine learning is difficult in light of challenges one and two.

Since there are few if any techniques to handle discrete categorical data in a time series regression or classification in supervised machine learning, this problem is challenging to solve. The systems and methods described herein provide a solution to a classification problem that provides a risk of an adverse event that is running as a time series machine learning model.

By freezing time and creating a classifier that determines whether a captured set of alerts within a window has an adverse event shown by data (e.g., remediation or fraud event), the classifier may be built outside of the time series problem by using positive and negative cases to give a prediction and a probability of a risk of an adverse event. This classifier may then be inserted into the time series data, and analyzed as if time has been rewound. The model may be further calibrated by looking at remediation events and tuning the model hyperparameters accordingly to increase accuracy. In doing so, the overarching technical problem may be split into two smaller problems: a classification problem and time series regression problem.

Accordingly, the systems and methods described herein provide numerous technical advantages. First, the model may be based on machine learning and might not be rule based. A hierarchical feature importance of alerts may be produced after the machine has been trained. In doing so, the machine learning technique makes the models dynamic (e.g., changes based on user alerts and user profile), scalable (e.g., can add new alerts and the system may account for new alert feature importance), and/or automated (e.g., not rule based—the machine learns what is best from the data).

Second, to understand the numerous alerts, a special graph may be created that is a time series graph plotting discrete alert data vs. time, along with a corresponding score. This graph may facilitate the process of understanding complex alert data and its relation to scoring.

Third, the small amount of data needed for machine learning may be addressed by aggregating large collections of users who have had positive and/or negative remediation events as positive and negative training cases.

Fourth, the time series classification problem may be addressed by splitting it into two smaller sub-problems.

Fifth, the classifier may be trained separately away from the time series component. The data may be trained upon and calibrated for the best results. To do so, a logistic regression classifier may be used (e.g., from the sci-kit learn library).

Sixth, the classification model may be re-inserted back into the time series, and a second round of model parameters may be optimized to produce the best accuracy. These parameters may include compounding (e.g., adding the impact of the same alert capture in the same window), N-day window capture, Tri-Label Encoding (e.g., treating alerts as three different types of categorical data based on a “me” or “not me” response from a user, or a “no response” from the user).

Accordingly, the machine learning model/computational technique described herein may produce a score correlating to adverse outcomes in the field of identity protection, and is automated, scalable to new alerts, dynamic, and effective for tiny amounts of data. The model may utilize user profile information and alert data for a user, and may be either predictive or explanatory. Further, the model may use a two-tier approach to split the primary technical challenge addressed herein into two sub problems: a classification problem and a time series, and addresses these problems using two calibration or training sessions separate in time and space (e.g., a classification training model session then a time series calibration session). Accordingly, the model performs alert compounding, N-day window alert capture, and/or tri-label alert encoding as techniques to improve accuracy. In addition, the model may produce a unique graph that plots discrete alert data vs. time, along with a corresponding score.

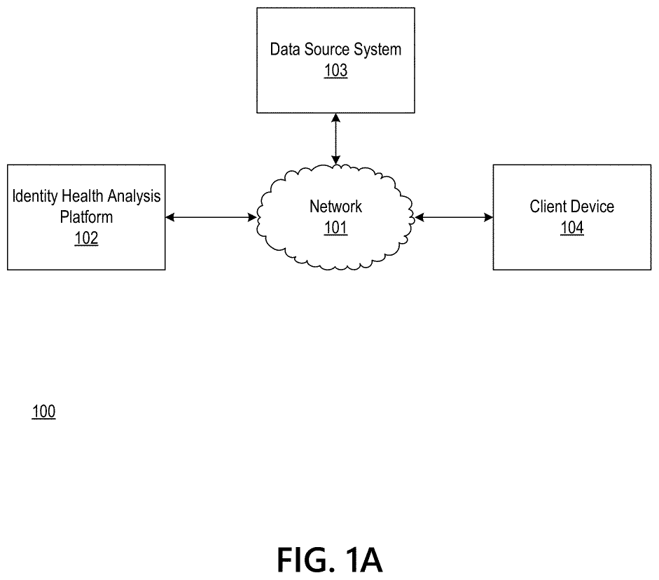

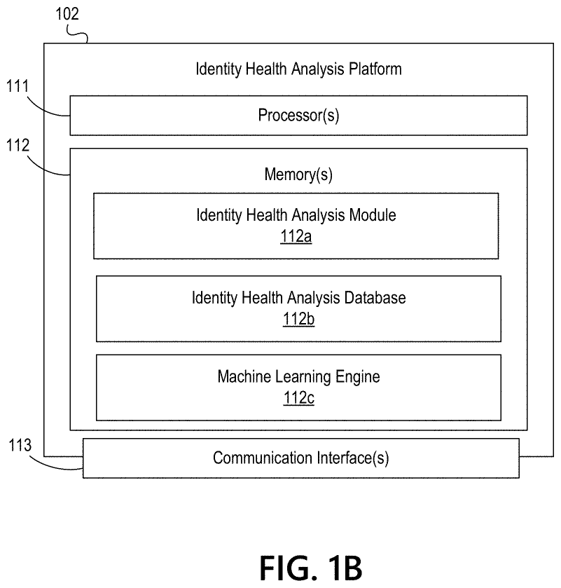

FIGS. 1A and 1B depict an illustrative computing environment for applying machine learning for identity health scoring in accordance with one or more example embodiments. Referring to FIG. 1A, computing environment 100 may include one or more computer systems. For example, computing environment 100 may include identity health analysis platform 102, data source system 103, and client device 104.

As illustrated in greater detail below, identity health analysis platform 102 may include one or more computing devices configured to perform one or more of the functions described herein. For example, identity health analysis platform 102 may include one or more computers (e.g., laptop computers, desktop computers, servers, server blades, or the like) and/or other computer components (e.g., processors, memories, communication interfaces). In addition, and as illustrated in greater detail below, identity health analysis platform 102 may be configured to apply one or more machine learning methods to train and deploy models for identity health analysis.

Data source system 103 may be or include one or more computing devices (e.g., servers, server blades, and/or other devices) configured to store historical and current fraud alert and/or user profile data. For example, data source system 103 may be configured to store fraud alerts sent to a user, user input indicating whether or not the fraud alerts correspond to an actual fraud event, whether or not user input was received indicating whether or not fraud alerts correspond to an actual fraud event, user profile information, and/or other identification health information.

Client device 104 may be a computing device (e.g., smartphone, tablet, desktop computer, laptop computer, or other personal computing device) that may be used by an enterprise user (e.g., a customer of an enterprise organization, such as an insurance provider). In some instances, the client device 104 may be used to display identity health information and/or other alerts/graphical user interfaces.

Computing environment 100 also may include one or more networks, which may interconnect one or more of identity health analysis platform 102, data source system 103, client device 104, and/or one or more other systems, public networks, sub-networks, and/or the like. For example, computing environment 100 may include a network 101.

In one or more arrangements, identity health analysis platform 102, data source system 103, client device 104, and/or the other systems included in computing environment 100 may be any type of computing device capable of receiving a user interface, receiving input via the user interface, and/or communicating the received input to one or more other computing devices. For example, the systems included in computing environment 100 may, in some instances, be and/or include server computers, desktop computers, laptop computers, tablet computers, smart phones, or the like that may include one or more processors, memories, communication interfaces, storage devices, and/or other components. As noted above, and as illustrated in greater detail below, any and/or all of identity health analysis platform 102, data source system 103, and/or client device 104 may, in some instances, be special-purpose computing devices configured to perform specific functions.

Referring to FIG. 1B, identity health analysis platform 102 may include one or more processors 111, memory 112, and communication interface 113. A data bus may interconnect processor 111, memory 112, and communication interface 113. Communication interface 113 may be a network interface configured to support communication between identity health analysis platform 102 and one or more networks (e.g., network 101, or the like). Memory 112 may include one or more program modules having instructions that when executed by processor 111 cause identity health analysis platform 102 to perform one or more functions described herein and/or one or more databases that may store and/or otherwise maintain information which may be used by such program modules and/or processor 111. In some instances, the one or more program modules and/or databases may be stored by and/or maintained in different memory units of identity health analysis platform 102 and/or by different computing devices that may form and/or otherwise make up identity health analysis platform 102. For example, memory 112 may have, store, and/or include an identity health analysis module 112a, an identity health analysis database 112b, and a machine learning engine 112c. Identity health analysis module 112a may have instructions that direct and/or cause identity health analysis platform 102 to train, maintain, and deploy a machine learning model for identity health analysis, as discussed in greater detail herein. Identity health analysis database 112b may store information (e.g., alert information, user input data labels, or the like) used by identity health analysis module 112a and/or identity health analysis platform 102 in training, maintaining, and deploying a machine learning model for identity health analysis and/or in performing other functions. Machine learning engine 112c may have instructions that direct and/or cause the identity health analysis platform 102 to identify and predict identity health information, and to set, define, and/or iteratively refine optimization rules, techniques and/or other parameters used by the identity health analysis platform 102 and/or other systems in computing environment 100.

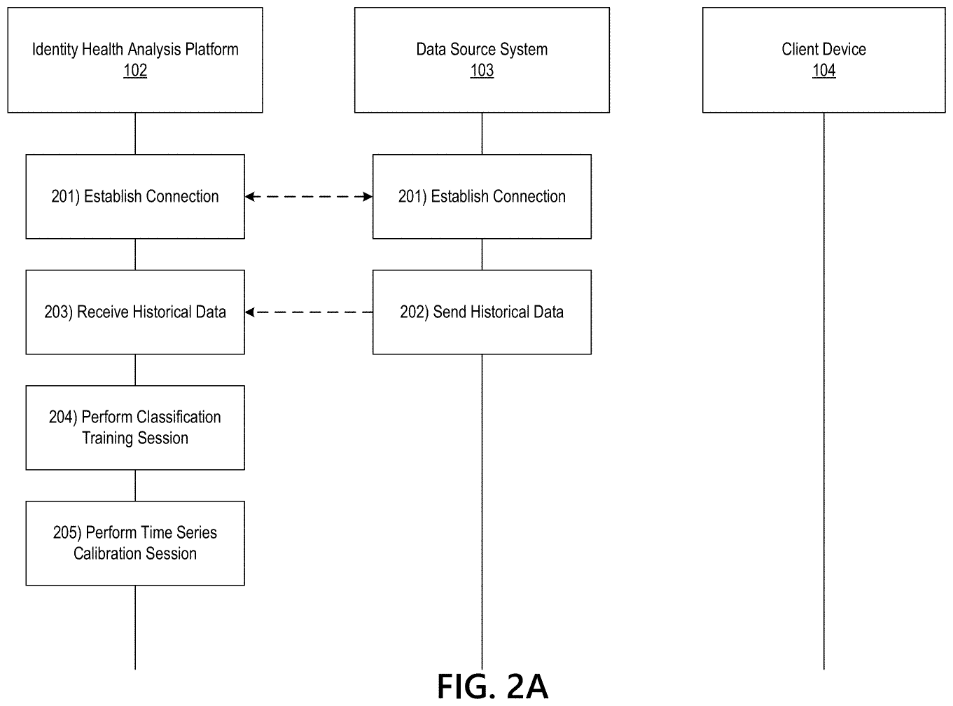

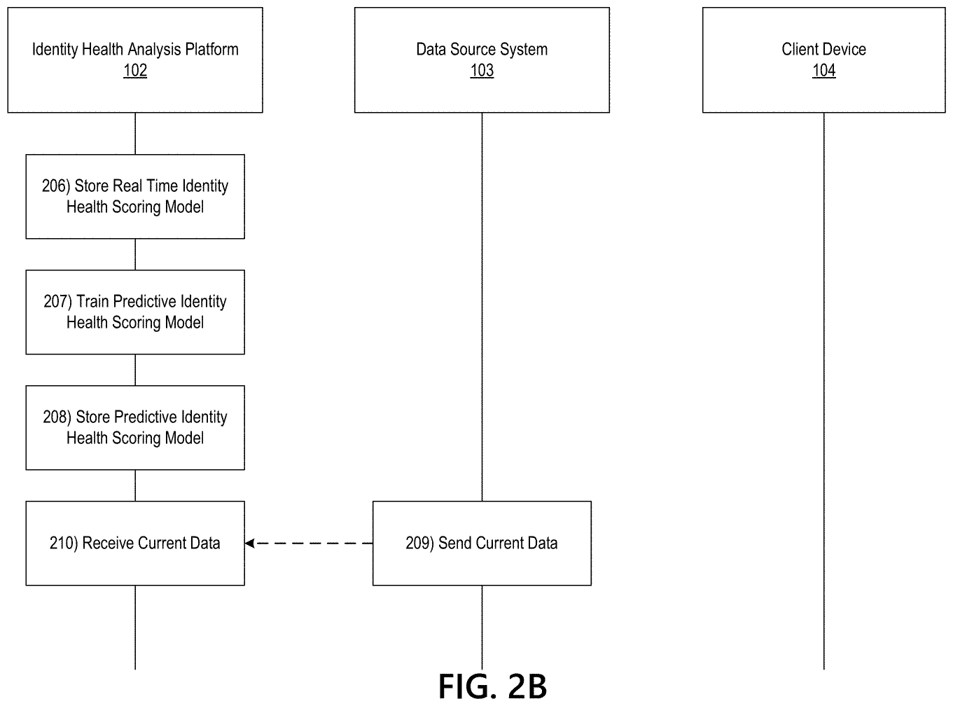

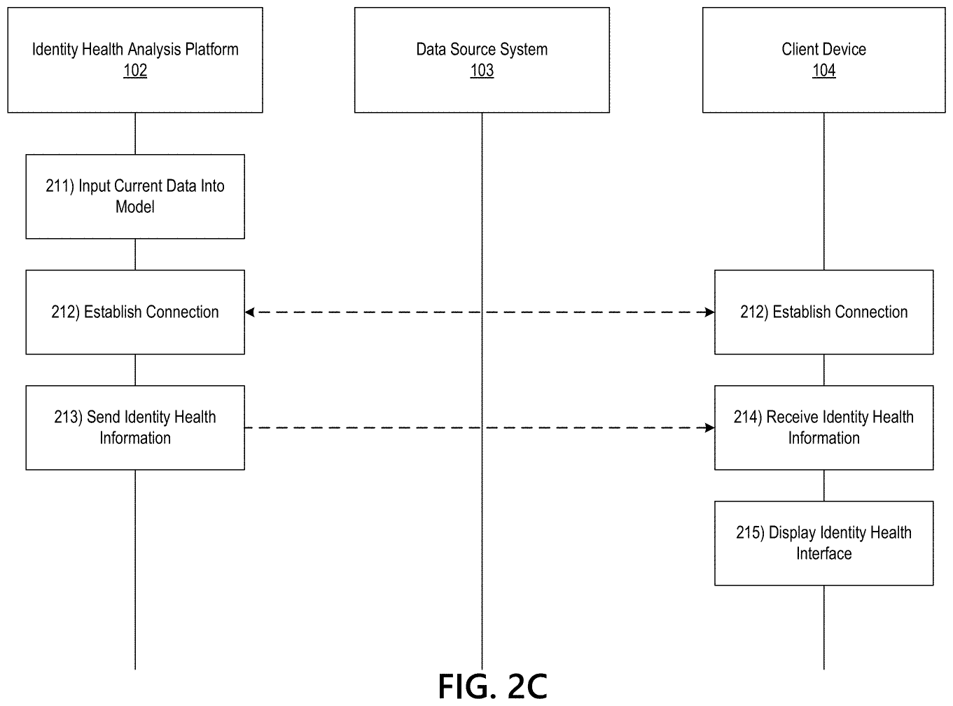

FIGS. 2A-2C depict an illustrative event sequence for applying machine learning for identity health scoring in accordance with one or more example embodiments. Referring to FIG. 2A, at step 201, the data source system 103 may establish a connection with the identity health analysis platform 102. For example, the data source system 103 may establish a first wireless data connection with the identity health analysis platform 102 to link the data source system 103 to the identity health analysis platform 102 (e.g., in preparation for sending historical data). In some instances, the data source system 103 may identify whether or not a connection is already established with the identity health analysis platform 102. If a connection is already established with the identity health analysis platform 102, the data source system 103 might not re-establish the connection. If a connection is not yet established, the data source system 103 may establish the first wireless data connection as described herein.

At step 202, the data source system 103 may send historical data (e.g., historical event information) to the identity health analysis platform 102. For example, the data source system 103 may send alert information (e.g., fraud alerts, and/or other types of alerts—e.g., not limited to just one alert at a time), timestamp information, user data labels (e.g., user input indicating whether or not an alert accurately identified fraud), user response information (e.g., whether a user failed to respond whether or not an alert accurately identified fraud), user profile information, and/or other identity health information. In some instances, in sending the historical data, the data source system 103 may send third party data (e.g., from a financial institution, credit card company, and/or other institution). In some instances, in sending the historical data, the data source system 103 may send data corresponding to a plurality of individuals. In some instances, the data source system 103 may send the historical data to the identity health analysis platform 102 while the first wireless data connection is established.

At step 203, the identity health analysis platform 102 may receive the historical data sent at step 202. For example, the identity health analysis platform 102 may receive the historical data via the communication interface 113 and while the first wireless data connection is established.

At step 204, the identity health analysis platform 102 may initiate training of a real time identity health analysis model. For example, the identity health analysis platform 102 may perform the first of two steps described herein to train the real time identity health analysis model. The identity health analysis platform 102 may perform a classification training session, where the identity health analysis platform 102 uses logical regression to train a classifier model based on the historical data. Additionally or alternatively, the identity health analysis platform 102 may utilize a sci-kit learn library to train the classifier model.

More specifically, the identity health analysis platform 102 may select a model for classification based on static data (e.g., the historical data). Once a model is selected, the identity health analysis platform 102 may create positive and negative cases for training (e.g., label the historical data as positive or negative). For example, in creating the positive cases, the identity health analysis platform 102 may identify alerts for which remediation occurred (e.g., a user indicated that an alert was accurate with a “not me” input—e.g., indicating that he or she did not make a particular transaction, and thus the alert properly flagged the transaction). In creating the negative cases, the identity health analysis platform 102 may identify alerts for which no remediation occurred (e.g., no user input was received regarding the alert or input received indicating that an alert incorrectly flagged a fraud event—e.g., by providing a “me” input confirming that the user actually performed a particular flagged transaction, account request, or the like). In these instances, the identity health analysis platform 102 may select alerts (corresponding to the historical data) for a given time range prior to a particular event (e.g., alert) being analyzed. For example, for positive cases, the identity health analysis platform 102 may select alerts from six months prior to an event being analyzed up to one day prior to the event being analyzed. For negative cases, the identity health analysis platform 102 may select alerts from twelve months prior to an event being analyzed up to six months prior to the event being analyzed. The identity health analysis platform 102 may then break this list of selected alerts into n sized sequences, which retain an original time order. Then, the identity health analysis platform 102 may accordingly train a time static classifier to distinguish between positive and negative cases.

For example, if training sequences of three alerts are being used within a five alert sequence (a, b, c, d, e), the training data would be as follows: (a), (a, b), (a, b, c), (b, c, d), (c, d, e), (d, e), (e). Accordingly, these sequences may be subsequently scored and aggregated.

At step 205, the identity health analysis platform 102 may perform the second of the two steps described herein to train the real time identity health analysis model. For example, the identity health analysis platform 102 may perform time series calibration using the trained classifier model as an input (e.g., the identity health analysis platform 102 may insert the time static classifier (e.g., trained at step 204) into time series data). In some instances, in performing the time series calibration, the identity health analysis platform 102 may tune parameters of the real time identity health analysis model by performing techniques including one or more of: alert compounding, N-day window alert capture, tri-label encoding, and/or other techniques.

For example, in performing the alert compounding, the identity health analysis platform 102 may count a particular alert each time that it appears in a capture window (e.g., if the same alert appears three times in a seven day capture window, it is counted three times). It may be important to consider the frequency of such alerts, as higher frequency may indicate higher risk (e.g., three “new accounts opened” alerts may be more significant than one).

With regard to N-day window alert capture, the identity health analysis platform 102 may consider alerts from a particular time duration for refinement of the real time health identity model (e.g., the window period for each calculation date). For example, if the calculation date is today, and a seven day window is being implemented, the identity health analysis platform 102 may go back seven days to capture alerts that have been sent, and may include these alerts in the time series calibration.

With regard to tri-label encoding, the historical data (e.g., alerts) is labeled with “me,” “not me,” or “no response” based on user responses to alerts that were sent to them. For example, a user may receive an alert that a transaction was executed, and may indicate that he or she executed the transaction. In other instances, the user may indicate that he or she did not execute the transaction (e.g., an identified fraud event). In other instances, the user may provide no indication of whether or not he or she executed the transaction. These responses may be mathematically modeled, by the identity health analysis platform 102, into the real time identity health analysis model.

By applying such techniques as N-day window capture and tri-label encoding, the identity health analysis platform 102 may increase accuracy of the real time identity health analysis model by more closely reflecting severity in the real world (the ground truth). This may result in a machine learning model configured to output identity health information for a user.

Referring to FIG. 2B, at step 206, the identity health analysis platform 102 may store the real time identity health scoring model, trained at steps 204 and 205, which may enable the real time identity health scoring model for future use. In some instances, the identity health analysis platform 102 may store the real time identity health scoring model in the memory 112 (e.g., identity health analysis module 112a, identity health analysis database 112b, and/or machine learning engine 112c).

At step 207, the identity health analysis platform 102 may train a predictive identity health scoring model. For example, in contrast to the real time identity health scoring model trained above at steps 204 and 205, which may be configured to identify a real time identity health, the predictive identity health scoring model may be configured to predict a future state of identity health for an individual. In some instances, the identity health analysis platform 102 may train the predictive identity health scoring model using neural network regression. In doing so, the identity health analysis platform 102 may pull a sequence of alerts from the historical data, and label them as positive or negative based on the presence or absence of a remediation event respectively (e.g., as described above with regard to the real time identity health scoring model).

In training the predictive identity health scoring model, the identity health analysis platform 102 might not remove duplicate alerts. For example, in a sequence of three alerts, a valid training sample for the identity health analysis platform 102 may include three identical alerts, and the identity health analysis platform 102 may consider each one. Furthermore, in training the predictive identity health scoring model, the identity health analysis platform 102 may consider alert data from an N-day window (e.g., a number of days back in time). For example, for alerts labeled as positive cases, the predictive identity health scoring model may use a seven day window prior to occurrence of the corresponding remediation events (e.g., alert data for a week prior to receipt of user input indicating “me” or “not me). For alerts labeled as negative cases, the predictive identity health scoring model may collect alerts for a seven day period that ends a week from the current date). Accordingly, for score prediction in a seven day model, the identity health analysis platform 102 may collect a week's worth of alerts. Seven days is merely an illustrative time window, and other windows (e.g., 6 months, or the like), may be used without departing from the scope of the disclosure.

At step 208, the identity health analysis platform 102 may store the predictive identity health scoring model trained at step 207, which may enable the predictive identity health scoring model for future use. In some instances, the identity health analysis platform 102 may store the predictive identity health scoring model in the memory 112 (e.g., identity health analysis module 112a, identity health analysis database 112b, and/or machine learning engine 112c).

At step 209, the data source system 103 may send current data (e.g., new event data) for a user to the identity health analysis platform 102. For example, the data source system 103 may send data such as alert information (e.g., fraud alerts), timestamp information, user data labels (e.g., user input indicating whether or not an alert accurately identified fraud), user response information (e.g., whether a user failed to respond whether or not an alert accurately identified fraud), user profile information, and/or other identity health information. In some instances, the data source system 103 may send the current data to the identity health analysis platform 102 while the first wireless data connection is established. In some instances, step 209 may be optional, and the data source system 103 might not send additional (current) data to the identity health analysis platform 102.

At step 210, the identity health analysis platform 102 may receive the current data sent at step 209. For example, the identity health analysis platform 102 may receive the current data via the communication interface 113 and while the first wireless data connection is established. In some instances, step 210 may be optional, and the identity health analysis platform 102 might not receive additional (current) data from the data source system 103.

Referring to FIG. 2C, at step 211, the identity health analysis platform 102 may input the current data into the real time identity health analysis model and/or the predictive identity health analysis model. In some instances, steps 209/210 may be optional, and thus rather than inputting additional (current) data into the real time identity health analysis model and/or the predictive identity health analysis model, the identity health analysis platform 102 may make a prediction based on the real time identity health analysis model and/or the predictive identity health analysis model itself.

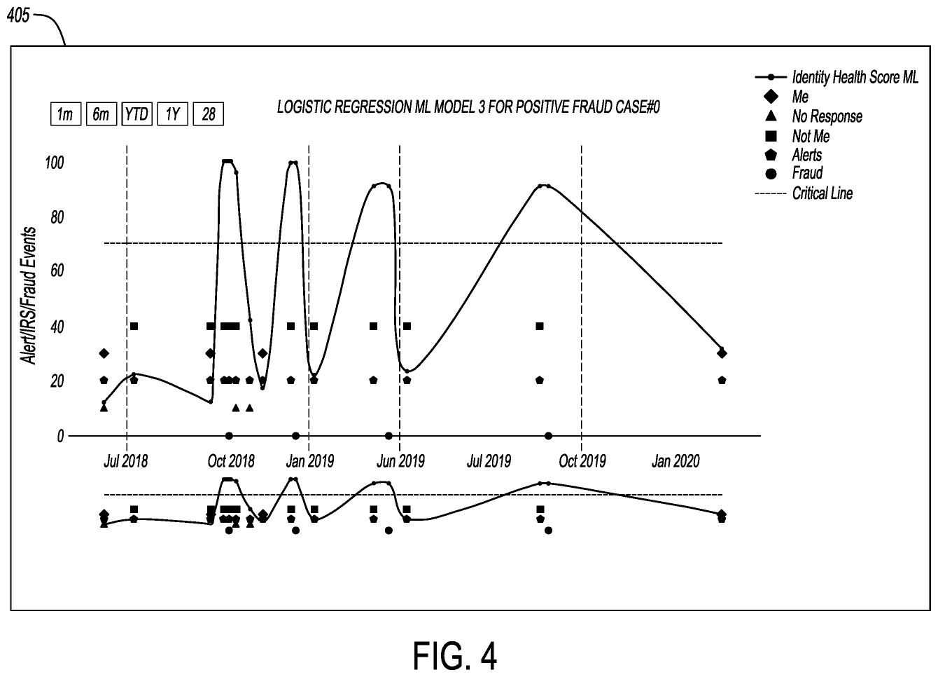

In some instances, the identity health analysis platform 102 may compute a series of identity health scores, which may be plotted on a graph (e.g., as shown in graphical user interface 405, which is illustrated in FIG. 4) as an identity health trend line. For example, the identity health analysis platform 102 may use the real time identity health analysis model and/or the predictive identity analysis model to identify values between 0 and 1 (e.g., representative of an identity health score for a particular moment in time), and multiplying these values by 100 to actually get the identity health scores. In some instances, the identity health analysis platform 102 may generate this graph and send it to the client device 104 for display. In other instances, the identity health analysis platform 102 may generate/send the scoring/other information that may be used by the client device 104 to produce the graph. As described below, the graph may include an identity health score trend for a user as plotted against time, along with information such as instances of alerts (e.g., fraud alerts), user data labels (e.g., user input indicating a response of “me” or “not me” for an alert), fraud events, and/or other information. In these instances, the identity health analysis platform 102 may generate an identity health score trend that peaks when a confirmed fraud event occurs. In some instances, the identity health analysis platform 102 may include a critical line on the graph, indicating a threshold level for concern (e.g., if the identity health score trend exceeds critical line, this may indicate poor identity health).

In some instances, the real time identity health analysis model and/or the predictive identity health analysis model may weight alerts differently in computation of the identity health scores. For example, the models may weight “new inquiry” alerts (e.g., indicating that a new inquiry was made on a credit file) differently than “financial transaction” alerts (e.g., indicating that a transaction above a given threshold was executed). In some instances, the models may further weight alerts based on user responses to the alerts (e.g., a lack of response, “me”/“not me” response (e.g., a user confirming whether or not a fraud alert is accurate), or the like). For example, the models may apply a weight value of 1 to identity health scores for alert type “new inquiry” and response “not me,” and a weight value of 0.925 to the identity health scores for alert type “new account” and response “not me.”

In doing so, the identity health analysis platform 102 may generate real time and/or predictive identity health information indicating a current and/or future status of the user's identity health.

At step 212, the identity health analysis platform 102 may establish a connection with the client device 104. For example, the identity health analysis platform 102 may establish a second wireless data connection with the client device 104 to link the identity health analysis platform 102 to the client device 104 (e.g., in preparation for sending identity health information). In some instances, the identity health analysis platform 102 may identify whether or not a connection is already established with the client device 104. If a connection is already established with the client device 104, the identity health analysis platform 102 might not re-establish the connection. If a connection is not yet established with the client device 104, the identity health analysis platform 102 may establish the second wireless data connection as described herein.

At step 213, the identity health analysis platform 102 may send the identity health information, generated at step 212, to the client device 104. For example, the identity health analysis platform 102 may send the identity health information to the client device 104 via the communication interface 113 and while the second wireless data connection is established. In some instances, along with the identity health information, the identity health analysis platform 102 may send one or more commands directing the client device 104 to display an identity health interface using the identity health information.

At step 214, the client device 104 may receive the identity health information sent at step 213. For example, the client device 104 may receive the identity health information while the second wireless data connection is established. In some instances, the client device 104 may also receive the one or more commands directing the client device 104 to display the identity health interface using the identity health information.

At step 215, based on or in response to the one or more commands directing the client device 104 to display the identity health interface, the client device 104 may generate and/or otherwise display the identity health interface based on the identity health information. For example, the client device 104 may display a graphical user interface similar to graphical user interface 405, which is illustrated in FIG. 4. For example, the client device 104 may display an identity health score trend for the user as plotted against time, along with information such as instances of alerts (e.g., fraud alerts), user data labels (e.g., user input indicating a response of “me” or “not me” for an alert), fraud events, and/or other information. In this example, the client device 104 may display an identity health score trend that peaks when a confirmed fraud event occurs. In some instances, the client device 104 may display a critical line on the graph, indicating a threshold level for concern (e.g., if the identity health score trend exceeds critical line, this may indicate poor identity health).

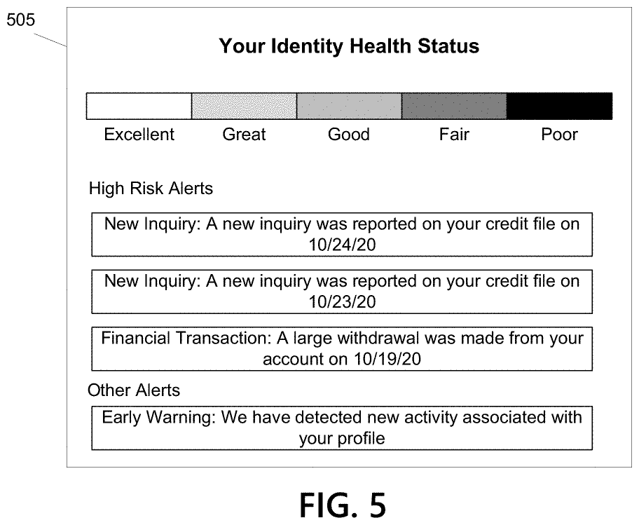

Additionally or alternatively, the client device 104 may display a graphical user interface similar to graphical user interface 505, which is illustrated in FIG. 5. For example, the client device 104 may display a graphical user interface indicating a sliding color scale, and a point on the scale that reflects the status of a user's identity health (e.g., excellent to poor). In this example, the client device 104 may reflect the user's identity health as a particular color instead of or in addition to a score. Additionally or alternatively, the client device 104 may display alerts related to identity health (e.g., new inquiries, financial transactions, early warnings, and/or other information).

In instances where a particular individual is not a customer of the enterprise organization corresponding to identity health analysis platform 102, the client device 104 might not display an interface that includes a score generated specifically for that individual based on their data (e.g., because the machine learning models described above might not have access to alert/event data for that individual). However, the client device 104 may display an interface that includes generalized identity health information for the individual based on a comparison of factors such as age, location, income, region, and/or other demographics information for the individual to individuals for whom data is stored in the models. Accordingly, a representative score may be generated for the individual, though it might not be as customized as the scores/interfaces presented to a customer. In these instances, the analysis used to create such interfaces may be performed at the identity health analysis platform 102 (e.g., using the identity health scoring models described above), and results of the analysis may be sent to the client device 104 for display. Similarly, such methods could be used to generate a baseline assessment of new customers prior to analyzing their fraud data.

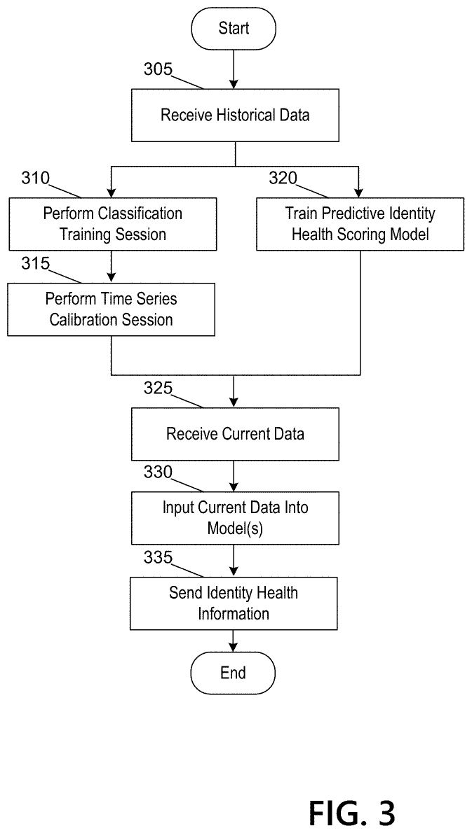

FIG. 3 depicts an illustrative method for applying machine learning for identity health scoring in accordance with one or more example embodiments. Referring to FIG. 3, at step 305, a computing platform having at least one processor, a communication interface, and a memory may receive historical data. At step 310, the computing platform may perform a classification training session for a real time identity health scoring model. At step 315, the computing platform may perform a time series calibration session for the real time identity health scoring model. At step 320, the computing platform may train a predictive identity health scoring model. At step 325, the computing platform may receive current data. At step 330, the computing platform may input the current data into the real time identity health scoring model and/or the predictive identity health scoring model to generate identity health information. At step 335, the computing platform may send identity health information and one or more commands directing a client device to display a graphical user interface based on the identity health information.

Although the systems and methods described herein primarily relate to identity health in the context of credit alerts, this is for illustrative purposes only, and such systems and methods may apply to other contexts such as identity theft, dark web sale of credentials, email compromise, social media compromise, and/or other contexts, without departing from the scope of this disclosure.

One or more aspects of the disclosure may be embodied in computer-usable data or computer-executable instructions, such as in one or more program modules, executed by one or more computers or other devices to perform the operations described herein. Generally, program modules include routines, programs, objects, components, data structures, and the like that perform particular tasks or implement particular abstract data types when executed by one or more processors in a computer or other data processing device. The computer-executable instructions may be stored as computer-readable instructions on a computer-readable medium such as a hard disk, optical disk, removable storage media, solid-state memory, RAM, and the like. The functionality of the program modules may be combined or distributed as desired in various embodiments. In addition, the functionality may be embodied in whole or in part in firmware or hardware equivalents, such as integrated circuits, application-specific integrated circuits (ASICs), field programmable gate arrays (FPGA), and the like. Particular data structures may be used to more effectively implement one or more aspects of the disclosure, and such data structures are contemplated to be within the scope of computer executable instructions and computer-usable data described herein.

Various aspects described herein may be embodied as a method, an apparatus, or as one or more computer-readable media storing computer-executable instructions. Accordingly, those aspects may take the form of an entirely hardware embodiment, an entirely software embodiment, an entirely firmware embodiment, or an embodiment combining software, hardware, and firmware aspects in any combination. In addition, various signals representing data or events as described herein may be transferred between a source and a destination in the form of light or electromagnetic waves traveling through signal-conducting media such as metal wires, optical fibers, or wireless transmission media (e.g., air or space). In general, the one or more computer-readable media may be and/or include one or more non-transitory computer-readable media.

As described herein, the various methods and acts may be operative across one or more computing servers and one or more networks. The functionality may be distributed in any manner, or may be located in a single computing device (e.g., a server, a client computer, and the like). For example, in alternative embodiments, one or more of the computing platforms discussed above may be combined into a single computing platform, and the various functions of each computing platform may be performed by the single computing platform. In such arrangements, any and/or all of the above-discussed communications between computing platforms may correspond to data being accessed, moved, modified, updated, and/or otherwise used by the single computing platform. Additionally or alternatively, one or more of the computing platforms discussed above may be implemented in one or more virtual machines that are provided by one or more physical computing devices. In such arrangements, the various functions of each computing platform may be performed by the one or more virtual machines, and any and/or all of the above-discussed communications between computing platforms may correspond to data being accessed, moved, modified, updated, and/or otherwise used by the one or more virtual machines.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, one or more of the steps depicted in the illustrative figures may be performed in other than the recited order, and one or more depicted steps may be optional in accordance with aspects of the disclosure.

What is claimed is:

1. A computing platform comprising:
at least one processor;
a communication interface communicatively coupled to the at least one processor; and
memory storing computer-readable instructions that, when executed by the at least one processor, cause the computing platform to:
train a machine learning model using historical event information, wherein training the machine learning model comprises:
classifying the historical event information using logical regression, and
after classifying the historical event information, performing time series calibration on the classified historical event information, wherein training the machine learning model configures the machine learning model to output identity health information;
receive new event information;
input the new event information into the machine learning model, wherein inputting the new event information into the machine learning model causes the machine learning model to output the identity health information; and
send, to a client device, the identity health information and one or more commands directing the client device to display an identity health interface, wherein sending the identity health information and one or more commands directing the client device to display the identity health interface causes the client device to display the identity health interface.

2. The computing platform of claim 1, wherein performing the time series calibration comprises performing one or more of alert compounding, N-day window alert capture, or tri-label encoding.

3. The computing platform of claim 2, wherein performing the alert compounding comprises considering an alert type a number of times that it appears within a capture window.

4. The computing platform of claim 2, wherein performing the N-day window alert capture comprises considering historical event information from N-days prior to a current date up to the current date.

5. The computing platform of claim 2, wherein the historical event information comprises identity threat alerts.

6. The computing platform of claim 5, wherein performing the tri-label encoding comprises:
labeling the historical event information based on user input indicating that an identity threat alert correctly identified a threat or incorrectly identified a threat, or
labeling the historical event information to indicate that user input was not received for the corresponding alert.

7. The computing platform of claim 1, wherein classifying the historical event information comprises:
graphing alert data and fraud event data against time, and
deriving, based on the graph, identity health score data.

8. The computing platform of claim 7, wherein displaying the identity health interface comprises displaying the graph.

9. The computing platform of claim 1, wherein displaying the identity health interface comprises displaying a color coded scale and a user's position on the color coded scale, wherein the user's position on the color coded scale indicates an identity health status for the user.

10. The computing platform of claim 1, wherein the machine learning model is configured to provide real time identity health scoring information.

11. The computing platform of claim 1, wherein the memory stores additional computer-readable instructions that, when executed by the at least one processor, further cause the computing platform to:
train, using neural network regression, a predictive identity health scoring model configured to predict identity threat events.

12. A method comprising:
at a computing platform comprising at least one processor, a communication interface, and memory:
training a machine learning model using historical event information, wherein training the machine learning model comprises:
classifying the historical event information using logical regression, and
after classifying the historical event information, performing time series calibration on the classified historical event information, wherein training the machine learning model configures the machine learning model to output identity health information;
applying the machine learning model to output the identity health information; and
sending, to a client device, the identity health information and one or more commands directing the client device to display an identity health interface, wherein sending the identity health information and one or more commands directing the client device to display the identity health interface causes the client device to display the identity health interface.

13. The method of claim 12, wherein performing the time series calibration comprises performing one or more of alert compounding, N-day window alert capture, or tri-label encoding.

14. The method of claim 13, wherein performing the alert compounding comprises considering an alert type a number of times that it appears within a capture window.

15. The method of claim 13, wherein performing the N-day window alert capture comprises considering historical event information from N-days prior to a current date up to the current date.

16. The method of claim 13, wherein the historical event information comprises identity threat alerts.

17. The method of claim 16, wherein performing the tri-label encoding comprises:
labeling the historical event information based on user input indicating that an identity threat alert correctly identified a threat or incorrectly identified a threat, or
labeling the historical event information to indicate that user input was not received for the corresponding alert.

18. The method of claim 12, wherein classifying the historical event information comprises:
graphing alert data and fraud event data against time, and
deriving, based on the graph, identity health score data.

19. The method of claim 18, wherein displaying the identity health interface comprises displaying the graph.

20. One or more non-transitory computer-readable media storing instructions that, when executed by a computing platform comprising at least one processor, a communication interface, and memory, cause the computing platform to:
train a machine learning model using historical event information, wherein training the machine learning model comprises:
classifying the historical event information using logical regression, and
after classifying the historical event information, performing time series calibration on the classified historical event information, wherein training the machine learning model configures the machine learning model to output identity health information;
receive new event information;
input the new event information into the machine learning model, wherein inputting the new event information into the machine learning model causes the machine learning model to output the identity health information; and
send, to a client device, the identity health information and one or more commands directing the client device to display an identity health interface, wherein sending the identity health information and one or more commands directing the client device to display the identity health interface causes the client device to display the identity health interface, wherein the identity health interface includes:
a graph of alert data plotted against time,
a graph of fraud data plotted against time, and
a graph of identity health score against time.
